(19) United States
(12) Patent Application Publication
(10) Pub. No.: US 2014/0328720 A1
(43) Pub. Date: Nov. 6, 2014

(54) PURIFICATION DEVICE AND PURIFICATION METHOD

(71) Applicant: NEC LIGHTING, LTD., Tokyo (JP)

(72) Inventors: Yasuhiro Mano, Tokyo (JP); Masami Kohno, Saitama (JP)

(21) Appl. No.: 14/358,434

(22) PCT Filed: Nov. 14, 2012
(86) PCT No.: PCT/JP2012/079490

§ 371 Date: May 15, 2014

(30) Foreign Application Priority Data
Nov. 15, 2011 (JP) 2011-249463

(51) Int. Cl. A61L 2/20; A61L 2/08; A61L 2/10

(52) U.S. Cl. CPC A61L 2/202; A61L 2/10; A61L 2/088
USPC 422/24; 422/186.12

(57) ABSTRACT

A purification apparatus includes an ultraviolet lamp (41) emitting an ultraviolet ray with a wavelength having ozone generating effect and an ultraviolet ray with a wavelength having sterilizing effect and an accommodator (43) accommodating the ultraviolet lamp (41) in an accommodation space thereinside. The accommodator (43) includes a transmission portion transmitting the ultraviolet ray with a wavelength having sterilizing effect. A gas supplied from an oxygen introducing portion (50) into the accommodation space contains oxygen. Thus, ozone is generated in the accommodation space due to the ozone generating effect of the ultraviolet ray emitted by the ultraviolet lamp (41). The ozone generated in the accommodation space is exhausted from an ozone exhausting portion (51).

### TECHNICAL FIELD

The present invention relates to a purification apparatus and a purification method.

### BACKGROUND ART

There has been proposed a nutrient solution culture system in which a nutrient solution (a culture solution) used for hydroponic culture in plant factories or the like is sterilized and purified by the actions of ozone, an ultraviolet ray, and a photocatalyst (for example, see Patent Literature 1). The nutrient solution culture system described in Patent Literature 1 includes a sterilization and purification unit for sterilizing and purifying a nutrient solution. The sterilization and purification unit is provided with electrodes. The electrodes are charged to high voltage to generate silent discharge, and a gas is allowed to pass through a discharge section, thereby generating ozone.

### CITATION LIST

### Patent Literature

Patent Literature 1: Unexamined Japanese Patent Application Kokai Publication No. 2009-247303

### SUMMARY OF INVENTION

### Technical Problem

However, when ozone is generated by the silent discharge system, the use of air as a raw material leads to the generation of NOx harmful to human bodies, and the use of oxygen as a raw material makes the structure of the apparatus complicated.

In addition, the nutrient solution culture system of Patent Literature 1 includes a flow channel for allowing the gas to pass through the discharge section, an ultraviolet light source, and the like, thus complicating the structure of the system.

The present disclosure has been accomplished in view of the above circumstances, and it is an objective of the present disclosure to provide an apparatus and a method for purification that have a simple structure and are safe.

### Solution to Problem

In order to achieve the objective, a purification apparatus according to the present disclosure comprises:

an ultraviolet lamp emitting an ultraviolet ray with a wavelength having ozone generating effect and an ultraviolet ray with a wavelength having sterilizing effect;
an accommodator including a transmission portion that transmits the ultraviolet ray with a wavelength having sterilizing effect and accommodating the ultraviolet lamp in an accommodation space inside the accommodator;
a gas supplier supplying an oxygen-containing gas into the accommodation space; and
an exhauster exhausting ozone generated in the accommodation space due to the ozone generating effect of the ultraviolet ray emitted by the ultraviolet lamp.

In order to achieve the objective, a purification method according to the present disclosure comprises:

a step of generating an ultraviolet ray with a wavelength having ozone generating effect and an ultraviolet ray with a wavelength having sterilizing effect;
a step of generating ozone by irradiating an oxygen-containing gas with the generated ultraviolet rays;
a step of supplying the generated ozone to a purification target; and
a step of irradiating the purification target with the generated ultraviolet rays.

### Advantageous Effects of Invention

The purification apparatus according to the present disclosure causes the generation of ozone without generating NOx. In addition, the ultraviolet ray with a wavelength having sterilizing effect transmits through the transmission portion to be emitted outside the accommodator. Accordingly, the present disclosure can safely perform purification in the simple structure.

### BRIEF DESCRIPTION OF DRAWINGS

FIG. 1 is a sectional view of a purification apparatus according to a first embodiment of the present disclosure;

FIG. 2 is a front view of an ultraviolet unit according to the first embodiment of the disclosure:

FIG. 3 is a sectional view of a seedling raising apparatus according to a second embodiment of the disclosure;

FIG. 4 is a sectional view of a purification apparatus according to a modification of the disclosure; and

FIG. 5 is a sectional view of a purification apparatus according to another modification of the disclosure.

### DESCRIPTION OF EMBODIMENTS

Hereinafter, embodiments of the present disclosure will be described with reference to the drawings. The same reference numerals are used throughout all the drawings to refer to the same elements. Additionally, in the description below, the terms “upper”, “lower”, “left”, and “right” will be used to refer to directions in accordance with the drawings for reference. However, it should be noted that these terms are merely used to facilitate understanding and do not limit the scope of the disclosure.

### First Embodiment

A purification apparatus according to a first embodiment of the present disclosure is an apparatus for purifying a liquid as a purification target. The term purification used herein includes all or a part of decomposition and removal of undesirable organic and inorganic compounds, killing or reduction of fungi, bacteria, viruses, and the like, suppression of growth of algae, and removal of malodors, and the like, mainly by oxidation decomposition reaction.

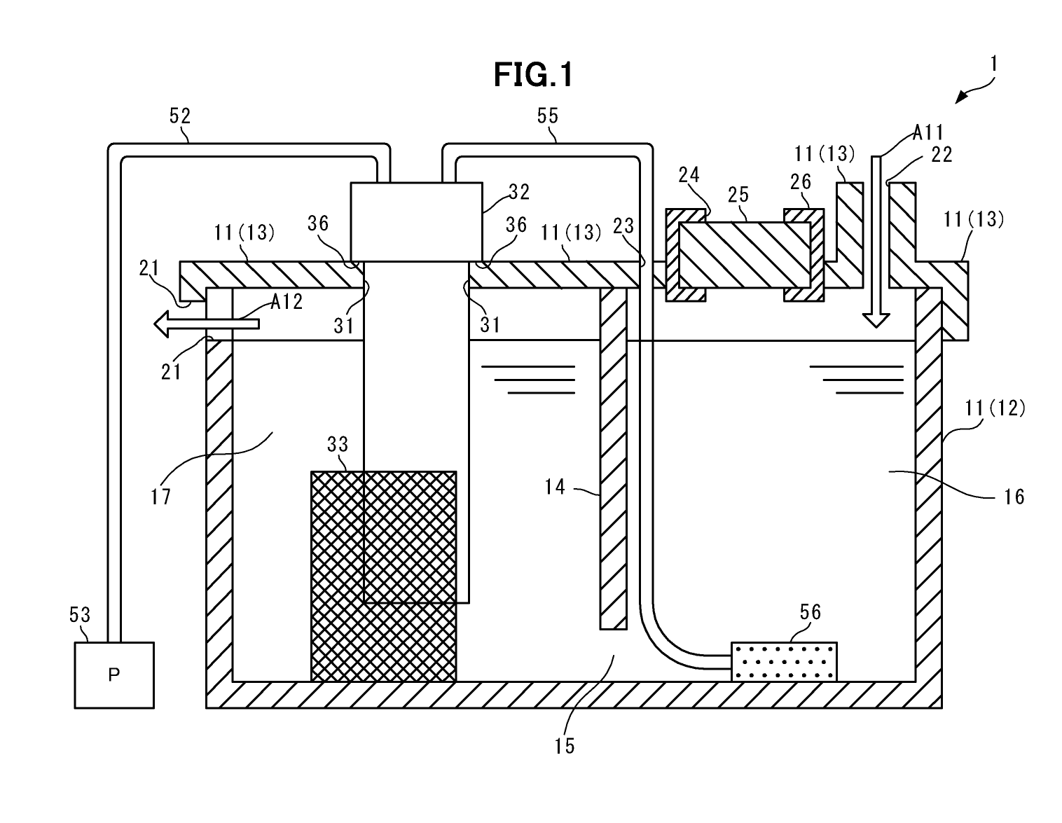

A purification apparatus 1 includes a rectangular parallelepiped purification container 11 including a purification space for allowing liquid to flow thereinside, as depicted in FIG. 1.

The purification container 11 includes a purification tank 12 that forms the purification space and whose upper part is open and a lid 13 provided on a top portion of the purification tank 12. Preferably, each of the purification tank 12 and the lid 13 is made of a material hardly corroded even when exposed to ozone and ultraviolet rays, which may be a metal (for example, stainless steel), a resin (for example, fluororesin), or glass.

The purification container 11 further includes a vertically extending tabular partition plate 14. The partition plate 14 is preferably made of a material hardly corroded even when exposed to ozone and ultraviolet rays, like the purification tank 12 and the lid 13, and the material may be a metal (for example, stainless steel), a resin (for example, fluororesin), or glass.

Both ends of the partition plate 14 (end portions thereof located in front and rear directions in FIG. 1) are closely fixed to inner walls of the purification tank 12 so that no gas or liquid may flow between the ends of the partition plate 14 and the purification tank 12. A top end portion of the partition plate 14 is closely adhered to the lid 13 so that no gas flows between the partition plate 14 and the lid 13

In order not to allow any gas or liquid to flow (in order to provide gas tightness or liquid tightness) between the members, a sealing member such as an 0-ring made of a material such as rubber or resin may be provided between the purification tank 12 and the partition plate 14, or rubber, resin, or the like may be applied therebetween. In addition, the purification tank 12 and the partition plate 14 may be press-bonded to each other with a screw or the like. Even in the following description, a sealing member or the like may be arranged between the gas-tightly or liquid-tightly provided members.

A bottom end portion of the partition plate 14 is apart from a bottom surface of the inside of the purification tank 12 by a predetermined distance. Accordingly, the purification space is partitioned into a first chamber 16 and a second chamber 17 communicating with each other via a lower flow passage 15 by the partition plate 14.

On a section of the lid 13 forming the first chamber 16 are provided an inflow inlet 22, a conduit portion 23, and a ventilator 24, respectively, forming an inflow channel, a conduit opening, and a ventilation opening, respectively, that communicate the first chamber 16 with the outside of the apparatus. The ventilator 24 includes a filter 25 for decomposing and ventilating ozone.

The filter 25 is disposed inside a filter attachment unit 26 that has the ventilator 24 and whose upper part is openable/closable for facilitating replacement. The filter attachment unit 26 is gas-tightly fixed to the lid 13. Preferably, the filter 25 is filled without any space in the ventilator 24 of the filter attachment unit 26 so that the first chamber 16 and the outside are ventilated only through the filter 25.

There is formed a cutout portion in an upper part of a section forming the second chamber 17 of the purification tank 12 and opposing to the partition plate 14. A top end portion of the purification tank 12 excluding the cutout portion is gas-tightly adhered to the lid 13. The cutout portion and a portion of the lid 13 opposing thereto form an outflow outlet 21 that is an outflow channel communicating the second chamber 17 with the outside.

In a section of the lid 13 forming the second chamber 17 is provided an ultraviolet unit installation portion 31 forming an installation opening that communicates the second chamber 17 with the outside. A substantially cylindrical ultraviolet unit 32 is inserted in the installation opening of the ultraviolet unit installation portion 31 in such a manner that a longitudinal direction of the unit is directed vertically. The ultraviolet unit 32 is gas-tightly disposed such that no gas flows between the unit and the ultraviolet unit installation portion 31.

The ultraviolet unit 32 has engagement portions 36 outwardly protruding at a top part of the unit. The engagement portions 36 are engaged with the lid 13 around the ultraviolet unit installation portion 31, whereby the ultraviolet unit 32 is located such that a lower part thereof is immersed in the second chamber 17, preferably in a liquid as a purification target flowing in the second chamber 17, by a predetermined length, as depicted in FIG. 1.

Around the lower part of the ultraviolet unit 32, namely, around the part thereof located in the second chamber 17 is provided a photocatalyst member 33 where photocatalytic reaction occurs due to ultraviolet rays and/or visible light.

The photocatalyst member 33 is a net-like member and includes, for example, a titanium oxide material, on a surface thereof. Such photocatalyst member 33 is produced, for example, by treating a surface of titanium metal with titanium oxide, applying a titanium oxide material on another material, or any other method.

The photocatalyst member 33 may be of a material causing photocatalytic effect by an ultraviolet ray (ranging from 100 to 400 nm) or visible light. Accordingly, the photocatalyst member 33 may be provided in a range where ultraviolet rays (ranging from 100 to 400 nm) and/or visible light emitted from the ultraviolet unit 32 can reach. Additionally, in order to provide the photocatalyst member 33 in the range where the ultraviolet rays and/or the visible light emitted from the ultraviolet unit 32 can reach, the photocatalyst member 33 may be attached to the ultraviolet unit 32 such that the photocatalyst member 33 is located in a predetermined range from the ultraviolet unit 32 through an attachment member or the like.

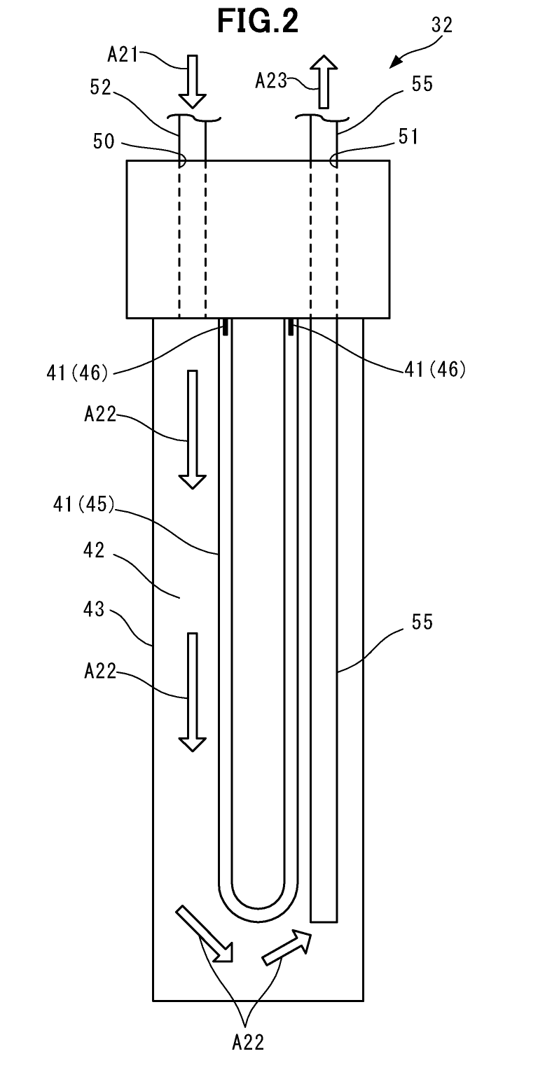

Hereinafter, a structure of the ultraviolet unit 32 will be described with reference to FIG. 2.

The ultraviolet unit 32 is a unit that externally emits ultraviolet rays to cause sterilizing effect and generate ozone. The ultraviolet unit 32 includes an ultraviolet lamp 41 that emits ultraviolet rays and an accommodator 43 including a gas-tight accommodation space 42 for accommodating the ultraviolet lamp 41 thereinside.

The ultraviolet lamp 41 is a lamp that emits light including an ultraviolet ray with a wavelength having ozone generating effect and an ultraviolet ray with a wavelength having sterilizing effect. The wavelength having ozone generating effect is, for example, 200 nm or shorter. The wavelength having sterilizing effect is, for example, around 260 nm (ranging from 200 to 320 nm).

The photocatalytic effect of the photocatalyst member 33 is caused by ultraviolet rays (ranging from 100 to 400 nm) and/or visible light in accordance with the material of the member, as described above. Accordingly, the ultraviolet ray with a wavelength having ozone generating effect and the ultraviolet ray with a wavelength having sterilizing effect also contribute to the photocatalytic effect of the photocatalyst member 33 depending on the material thereof. Additionally, when the light emitted by the ultraviolet lamp 41 includes an ultraviolet ray with a wavelength other than wavelengths having ozone generating effect and sterilizing effect and visible light, light with wavelengths thereof contribute to the photocatalytic effect of the photocatalyst member 33 depending on the material thereof.

The ultraviolet lamp 41 is, for example, a low-pressure ultraviolet lamp, which comprises a hot-cathode lamp, a cold-cathode lamp, an external electrode lamp, an electrodeless lamp, or the like.

The ultraviolet lamp 41 comprises, for example, a cold cathode lamp provided with a U-shaped tube 45 for sealing a gas and Ni cup electrodes 46 as electrodes for discharging electricity inside the U-shaped tube 45. For example, mercury and a mixed gas of Ne and Ar are sealed in the U-shaped tube 45. The sealed mixed gas has a pressure of, for example, approximately from 2.67 to 13.33 kPa (20 to 100 Torr), and preferably around 5.33 kPa (40 Torr). The mercury may be sealed such that the lamp during lighting has a mercury vapor pressure of, for example, 100 Pa or less.

By employing such a mercury lamp as the ultraviolet lamp 41, the ultraviolet lamp 41 emits light including an ultraviolet ray with a wavelength of approximately 185 nm having ozone generating effect, an ultraviolet ray with a wavelength of approximately 254 nm having sterilizing effect, and visible light.

The U-shaped tube 45 transmits both of the ultraviolet ray with a wavelength having ozone generating effect and the ultraviolet ray with a wavelength having sterilizing effect. Specific examples of a material of the U-shaped tube 45 include synthetic quartz, molten quartz, and a borosilicate glass containing at least one of Al, Na, K, Li, Ca, and Ba. Particularly, a borosilicate glass containing at least one of Al, Na, K, Li, Ca, and Ba is preferable as the material of the U-shaped tube 45 than quartz glass in terms of facilitating processing.

The accommodator 43 transmits the ultraviolet rays and visible light emitted by the ultraviolet lamp 41. Specific examples of a material of the accommodator 43 include synthetic quartz, molten quartz, ozoneless quartz, and a borosilicate glass containing at least one of Al, Na, K, Li, Ca, and Ba.

Of the ultraviolet rays emitted by the ultraviolet lamp 41, preferably, the accommodator 43 blocks the ultraviolet ray with a wavelength having ozone generating effect and transmits the ultraviolet ray with a wavelength having sterilizing effect. For example, the accommodator 43 is preferably made of a material such as ozoneless quartz.

Although omitted in the drawing, the accommodator 43 includes wires for supplying electric power to the electrodes 46 of the ultraviolet lamp 41, which are connected to the electrodes 46 of the ultraviolet lamp 41. In addition, an inverter or the like may be provided inside or outside the accommodator 43, although omitted in the drawing.

At a top part of the accommodator 43 are provided an oxygen introducing portion 50 and an ozone exhausting portion 51, respectively, forming an oxygen introducing opening and an ozone exhausting opening, respectively, that communicate the accommodation space 42 with the outside.

One end of an introduction pipe 52 made of, for example, silicon is gas-tightly attached to the oxygen introducing portion 50 as a gas supplier, and a pump 53 (see FIG. 1) is attached to the other end thereof. The pump 53, for example, electrically raises the pressure of air and sends out the air, whereby the air is introduced into the accommodation space 42 through the introduction pipe 52 and then the oxygen introducing portion 50.

As depicted in FIG. 2, an exhaustion pipe 55 made of, for example, silicon is inserted in the ozone exhausting portion 51 as an exhauster in a state of maintaining gas tightness between the pipe 55 and the ozone exhausting portion 51. One end of the exhaustion pipe 55 extends to a lower part of the accommodation space 42, and the other end thereof is attached to an air stone 56 as an ozone discharger, as depicted in FIG. 1. The air stone 56 is a member that converts an introduced gas into small air bubbles having a size of from around a few tens of nanometers to a few millimeters and discharges the air bubbles into a liquid, and is placed on the bottom of the first chamber 16.

Next, a description will be given of a method for purifying a liquid by the purification apparatus 1 having the above structure, with reference to FIGS. 1 and 2.

The liquid as the purification target flows from the inflow inlet 22 into the first chamber 16 of the purification container 11 as indicated by an arrow A11, then passes through the flow channel 15 into the second chamber 17, and flows outside from the outflow outlet 21 as indicated by an arrow A12.

In addition, as depicted in FIG. 2, air pressurized by the pump 53 is introduced into the accommodation space 42 through the introduction pipe 52 and the oxygen introducing portion 50 as indicated by an arrow A21, and then flows as indicated by an arrow A22. In the accommodation space 42, oxygen in the air is converted to ozone due to the action of the ultraviolet ray with a wavelength having ozone generating effect emitted from the ultraviolet lamp 41. The ozone generated in the accommodation space 42 is exhausted from the ozone exhausting portion 51 to the exhaustion pipe 55, as indicated by an arrow A23. The exhausted ozone is guided to the air stone 55 through the exhaustion pipe 55 and formed into small air bubbles to be discharged from the air stone 56 in the first chamber 16.

Accordingly, the liquid flown into the first chamber 16 is exposed to ozone before reaching the flow channel 15. Since the ozone is in the form of small air bubbles, a contact area between the ozone and the liquid is large. As a result, the liquid flowing in the first chamber 16 can be efficiently purified by the action of the ozone.

A part of the ozone is dissolved into the liquid in the first chamber 16. The remaining part of the ozone having not been dissolved into the liquid and then having reached a liquid surface in the first chamber 16 is exhausted outside through the filter 25 disposed in the ventilator 24 of the lid 13. At the time of exhaustion, the ozone is decomposed by the action of the filter 25 to be rendered harmless down to a level that is harmless at least to human bodies.

The liquid containing the ozone that has been dissolved in the first chamber 16 passes though the flow channel 15 and flows into the second chamber 17. The liquid as ozone water flown into the second chamber 17 is irradiated with the ultraviolet rays from the ultraviolet lamp 41 arranged in the second chamber 17. The accommodator 43 is made of a material transmitting at least the ultraviolet ray with a wavelength having sterilizing effect, as described above. Thus, the liquid is purified in the second chamber 17 due to the action of the ultraviolet ray transmitted through the accommodator 43.

The liquid in the second chamber 17 contains the dissolved ozone, and a part of the ozone is converted to OH radicals and O radicals by the at least one ultraviolet ray applied from the ultraviolet unit 32. Therefore, the liquid in the second chamber 17 is also purified by the actions of the OH radicals and the O radicals that are more strongly oxidative and more highly reactive than ozone.

Furthermore, the irradiation of the photocatalyst member 33 with the at least one ultraviolet ray and visible light from the ultraviolet unit 32 generates electrons and positive holes on a surface of the photocatalyst member 33. Thus, the liquid in the second chamber 17, particularly the liquid flowing near the photocatalyst member 33 is also purified by the actions of the electrons and the positive holes generated on the surface of the photocatalyst member 33.

The liquid purified in the second chamber 17 flows outside through the outflow outlet 21, as indicated by the arrow A12.

In this manner, the liquid flown in from the inflow inlet 22 is purified in the first chamber 16 and then purified by the strong purification power in the second chamber 17. As a result, many bacteria, viruses, fungi, and the like contained in the liquid before purification die and odors included in the liquid before purification are removed, so that transparency of the liquid improves more than before purification.

According to the present embodiment, since the ultraviolet lamp 41 exhibits both of the ozone generating effect and the sterilizing effect, it is unnecessary to additionally provide an ozone generating device. Thus, the structure of the purification apparatus can be simplified, as a result of which the purification apparatus 1 can be made compact at low cost.

The ultraviolet unit 32 of the purification apparatus 1 of the present embodiment generates ozone by the ultraviolet rays. Accordingly, NOx is not generated even with the use of air as a raw material, so that ozone can be safely generated even when air is used as the raw material. In addition, the use of air as the raw material can achieve low running cost.

Furthermore, in the present embodiment, the purification space is partitioned into the first chamber 16 and the second chamber 17, and the liquid as the purification target is exposed to ozone in the first chamber 16. Accordingly, since the liquid as the purification target can be sufficiently exposed to the ozone, the liquid can be purified by the ozone, as well as much ozone can be dissolved in the liquid to thereby improve the purification power in the second chamber 17.

In general, highly-concentrated ozone is harmful to human bodies. For example, the Japan Association of Industrial Health stipulates that the permissible concentration of ozone is 0.1 ppm. In the present embodiment, the purification space is substantially gas-tightly partitioned into the first chamber 16 and the second chamber 17 as described above, and ozone is discharged in the first chamber 16. Thus, ozone having not dissolved in the liquid in the first chamber 16 is decomposed through the filter 25 and then exhausted outside the purification apparatus 1. Accordingly, there is no exhaustion of ozone at a concentration high enough to be harmful to human bodies outside the purification apparatus 1, so that the purification apparatus 1 can utilize the purification effect of ozone and simultaneously can provide high safety.

While the first embodiment of the disclosure has been described hereinabove, the present embodiment is not limited thereto.

For example, although the lid 13 is an integral lid that shields the upper parts of the first and the second chambers 16 and 17, the lid 13 may comprise separate lids that shield each of the upper parts of the first and the second chambers 16 and 17.

The lid 13 may be provided only on the upper part of the first chamber 16. In this case, for example, the ultraviolet unit 32 may be fixed to the purification tank 12 by an attachment tool or may be placed on the bottom of the purification tank 12.

The inflow inlet 22 may be provided not on the lid 13 but, for example, on a side wall of the purification tank 12 forming the first chamber 16. For example, a pump or the like may be used for allowing the liquid of the purification apparatus 1 to flow in and flow out. However, in order to allow the liquid to flow in and flow out by a natural stream without using a pump or the like, an upper surface of the flowing liquid is substantially defined by the outflow channel. Accordingly, in this case, the inflow inlet 22 may be provided so as to be located upper than the outflow channel.

The partition plate 14 according to the present embodiment is an example of a partition portion. The partition portion can be any as long as the portion is a member that forms the flow channel 15 in the lower part of the tank 12 and partitions the purification space into the first and the second chambers 16 and 17, and is not limited to a plate-shaped member. In addition, the purification tank 12 may comprise, as separate bodies, a first purification tank and a second purification tank. In this case, the first purification tank may form the first chamber 16, the second purification tank may form the second chamber 17, and a flow tube for connecting the first purification tank with the second purification tank may form the flow channel 15 communicating the first chamber 16 with the second chamber 17.

In the present disclosure, it has been described that the ultraviolet lamp 41 includes the U-shaped tube 45. However, as an alternative to the U-shaped tube 45, a tube having an arbitrary shape, such as a straight tube, an L-shaped tube, or a W-shaped tube, may be used. In addition, the ultraviolet lamp 41 may comprise a plurality of tubes having an arbitrary shape, such as U-shaped tubes 45, straight tubes, L-shaped tubes, or W-shaped tubes. Efficient accommodation of the ultraviolet lamp 41 in the accommodation space 42 can increase an amount of light emitted from the ultraviolet lamp 41 without changing the size of the ultraviolet lamp 41. In this manner, the purification ability of the purification apparatus 1 can be improved and the purification apparatus 1 can be made compact.

In the ultraviolet lamp 41 used in the present embodiment, a fluorescent material may be applied on a part of the U-shaped tube 45 forming the ultraviolet lamp 41. The fluorescent material may have properties changing the wavelength of an ultraviolet ray mainly to that of visible light or may have properties mainly changing the wavelength of an ultraviolet ray (for example, from 254 to 350 nm).

In this case, the ultraviolet lamp 41 emits, together with the ultraviolet rays, an ultraviolet ray and visible light with wavelengths specific to the applied fluorescent material. When the accommodator 43 also transmits visible light, the photocatalyst member 33 causes a photocatalytic reaction due to the ultraviolet rays and the visible light.

The present embodiment has described the case in which among light emitted from the ultraviolet lamp 41, the accommodator 43 allows the ultraviolet ray with a wavelength having sterilizing effect and the ultraviolet ray with a wavelength having photocatalytic effect to pass through. However, the entire part of the accommodator 43 does not have to have such transmission properties. It is enough for the accommodator 43 to only partially include a transmission portion that transmits the ultraviolet ray with a wavelength having sterilizing effect and the ultraviolet ray with a wavelength having photocatalytic effect among the light emitted from the ultraviolet lamp 41.

In this case, the transmission portion is provided at a position where the portion is immersed in the liquid flowing in the purification tank 12, for example, in the lower part of the accommodator 43. When the upper surface of the flowing liquid is defined by the outflow channel, it is preferable to provide the transmission portion at a position lower than the outflow channel, since irradiation with light in the liquid can improve the purification effects by the ultraviolet rays and the dissolved ozone. In addition, the photocatalyst member 33 is irradiated with strong light, so that the purification effect by photocatalytic reaction can be improved.

Additionally, more preferably, the part of the accommodator 43 lower than the outflow channel is entirely used as the transmission portion. Irradiation with much more light in the liquid can improve the purification effects by the ultraviolet rays and the dissolved ozone. In addition, since the photocatalyst member 33 is irradiated with much more strong light, the purification effect by photocatalytic reaction can be improved.

When the photocatalytic member 33 is not provided, it is enough for the accommodator 43 to include a transmission portion made of a material transmitting the ultraviolet ray with a wavelength having sterilizing effect among the light emitted from the ultraviolet lamp 41. In other words, the accommodator 43 may be entirely made of such a material or may only partially include a transmission portion made of such a material.

When the accommodator 43 transmits the ultraviolet ray with a wavelength having ozone generating effect, ozone, although in small amount, can be generated even in the second chamber 17. When ozone is generated in the second chamber 17, much of the ozone is exhausted outside the purification apparatus 1 through the outflow outlet 21.

In general, ozoneless quartz has properties that transmit an ultraviolet ray with a wavelength having sterilizing effect and visible light and block an ultraviolet ray with a wavelength having ozone generating effect. Accordingly, the use of ozoneless quartz as a material of the accommodator 43 can suppress the generation of ozone in the second chamber 17 and can further reduce the possibility of ozone exhaustion outside the purification apparatus 1. Therefore, safety on the purification apparatus 1 can be improved.

On an outer surface of the accommodator 43 may be arranged a material having photocatalytic effect at a level that does not inhibit the transmission of the ultraviolet ray with a wavelength having sterilizing effect emitted from the ultraviolet lamp 41. For example, the surface of the accommodator 43 may be coated with a material having photocatalytic effect, such as titanium oxide.

When the outer surface of the accommodator 43 is provided with a material having photocatalytic effect, the photocatalytic effect of the material can purify a nutrient solution near the accommodator 43, as well as can prevent the adhesion of contaminants, such as algae and fungi, on the outer surface of the accommodator 43.

The gas introduced into the accommodation space 42 through the oxygen introducing portion 50 is not limited to air and can be any as long as the gas is an oxygen-containing gas. For example, in order to introduce highly concentrated oxygen into the accommodation space 42, an oxygen cylinder may be disposed that can adjust exhaust pressure, instead of the pump 53.

In the present embodiment, the ozone exhausting portion 51 is provided on the top part of the accommodator 43. However, the ozone exhausting portion 51 may be provided on the lower part of the accommodator 43. In this case, one end of the exhaustion pipe 55 may be attached to the ozone exhausting portion 51.

Furthermore, in the present embodiment, the ozone discharger comprises the air stone 56, but the structure of the ozone discharger is not limited thereto. For example, the ozone discharger may comprise a micro-bubble generating device that generates micro bubbles of ozone exhausted from the ozone exhausting portion 51 and a member that discharges the micro bubbles generated by the micro-bubble generating device into the liquid.

The discharge of ozone micro bubbles can further increase the contact area between the ozone and the liquid as compared to the use of the air stone 56 alone, as a result of which the purification effect by the ozone in the first chamber 16 can be improved. In addition, the liquid containing much more dissolved ozone is allowed to flow into the second chamber 17, so that the purification effect derived from the dissolved ozone in the second chamber 17 can be increased.

### Second Embodiment

The present embodiment will be described using an example of the purification apparatus 1 according to the first embodiment applied to hydroponic culture.

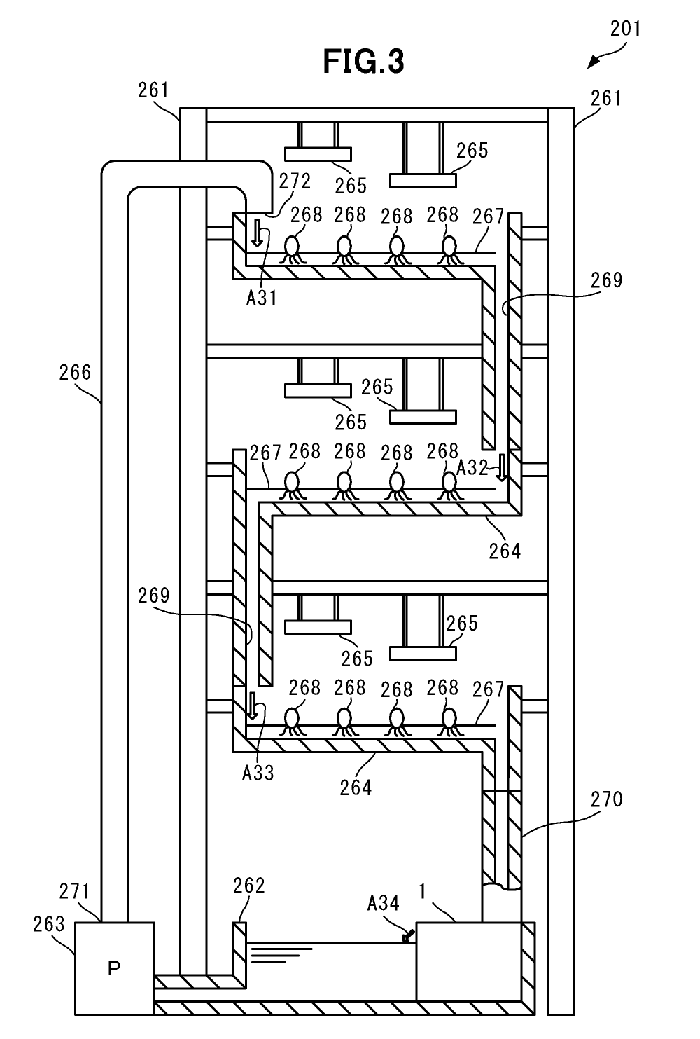

A seedling raising apparatus 201 according to a second embodiment of the present disclosure includes support members 261, a nutrient solution tank 262 inside which the purification apparatus 1 is installed, and a nutrient solution pump 263, as depicted in a schematic sectional view of FIG. 3. The support members 261, the nutrient solution tank 262, and the nutrient solution pump 263 are, for example, disposed on a floor.

The support members 261 support three-stage cultivation shelves 264, lighting fixtures 265 provided above each of the cultivation shelves 264, and a purified-nutrient solution pipe 266 extending from the nutrient solution pump 263 to a left end portion of an upper-stage cultivation shelf 264.

On each of the cultivation shelves 264 is provided a plant placement plate 267 having a plurality of holes. Plants 268 are placed on the plant placement plate 267. The height of each of the lighting fixtures 265 arranged above the each cultivation shelf 264 can be adjusted to apply light with an optimum intensity in accordance with growth states of the plants 268, and the like.

The upper-stage cultivation shelf 264 is provided with a nutrient solution conduit 269 extending downward from a right end portion of a bottom thereof toward a right end portion of a middle-stage cultivation shelf 264. The middle-stage cultivation shelf 264 includes a nutrient solution conduit 269 extending downward from a left end portion of a bottom thereof toward a left end portion of a lower-stage cultivation shelf 264. The lower-stage cultivation shelf 264 includes a contaminated-solution pipe 270 extending from a right end portion of a bottom thereof to the inflow inlet 22 of the purification apparatus 1 provided below the shelf.

The nutrient solution tank 262 is a tank that stores a nutrient solution including a nutrient for cultivating the plants 268. The nutrient solution pump 263 sucks and pressurizes the nutrient solution stored in the nutrient solution tank 262 and ejects the nutrient solution from a pressurized-solution ejecting portion 271 to the purified-nutrient solution pipe 266. The purified-nutrient solution pipe 266 is a pipe that guides the nutrient solution ejected from the nutrient solution pump 263 to the left end portion of the upper-stage cultivation shelf 264. One end of the purified-nutrient solution pipe 266 is connected to the pressurized-solution ejecting portion 271 of the nutrient solution pump 263 and the other end thereof has an injection portion 272 forming a downward opening. The injection portion 272 is provided above the left end portion of the upper-stage cultivation shelf 264.

Next, a description will be given of the flow of the nutrient solution in the seedling raising apparatus 201.

The nutrient solution is poured into the left end portion of the upper-stage cultivation shelf 264 from the injection portion 272, as indicated by an arrow A31. The nutrient solution flows in a right direction while sequentially contacting with roots of the plants 268 extending downward through the holes of the plant placement plate 267 provided on the upper-stage cultivation shelf 264.

The nutrient solution having reached the right end portion of the upper-stage cultivation shelf 264 flows to the right end portion of the middle-stage cultivation shelf 264 through the nutrient solution conduit 269, as indicated by an arrow A32. The nutrient solution furthermore flows in a left direction while sequentially contacting with roots of the plants 268 aligned on the middle-stage cultivation shelf 264.

The nutrient solution having reached the left end portion of the middle-stage cultivation shelf 264 flows to the left end portion of the lower-stage cultivation shelf 264 through the nutrient solution conduit 269, as indicated by an arrow A33. The nutrient solution furthermore flows in a right direction while sequentially contacting with roots of the plants 268 aligned on the lower-stage cultivation shelf 264.

The nutrient solution having reached the right end portion of the lower-stage cultivation shelf 264 is guided into the inflow inlet 22 of the purification apparatus 1 through the contaminated-solution pipe 270. The nutrient solution flown into the purification apparatus 1 from the inflow inlet 22 is purified by the actions of ozone, the ultraviolet rays, and the photocatalyst member 33, as described in the first embodiment, and then flows out from the outflow outlet 21, as indicated by an arrow A34.

The nutrient solution flown out from the outflow outlet 21 is received in the nutrient solution tank 262 and pressurized by the nutrient solution pump 263 to ascend through the purified-nutrient solution pipe 266 and to be poured again into the left end portion of the upper-stage cultivation shelf 264 from the injection portion 272. In this manner, the nutrient solution circulates through the cultivation shelves 264 and the purification apparatus 1.

In general, bacteria, fungi, algae, organic matter, and the like in the nutrient solution can be causes of diseases of the plants 268, can adhere to the roots of the plants 268 to inhibit the growth thereof, and also can cause offensive odors. In addition, in a case of using the plants 268 for food, the growth of pathogenic bacteria such as Escherichia coli in the solution can threaten food safety.

In order to prevent these problems, the nutrient solution is replaced accordingly, and the members to be brought in contact with the nutrient solution, such as the purified-nutrient solution pipe 266, the cultivation shelves 264, the nutrient solution conduit 269, the contaminated-solution pipe 270, and the nutrient solution tank 262, are cleaned as needed.

In the present embodiment, the purification apparatus 1 purifies the nutrient solution during the circulation of the solution. Thus, the frequencies of replacement of the nutrient solution and cleaning of the members contacted with the nutrient solution can be reduced, so that hydroponic culture can be economically carried out with little time and effort.

As described above, bacteria, fungi, algae, organic matter, and the like adhering to the roots of the plants 268 can inhibit the roots thereof from absorbing the nutrient. The present embodiment reduces bacterial, fungi, algae, organic matter, and the like adhering to the roots of the plants 268, whereby the plants 268 can sufficiently continue to absorb the nutrient and therefore the embodiment can improve the growth rate of the plants 268.

Ozone, after purifying the nutrient solution in the second chamber 17 of the purification apparatus 1, turns to oxygen. Then, a part of the ozone is dissolved into the nutrient solution, and then the nutrient solution flows out from the outflow outlet 21. Accordingly, the nutrient solution flown out from the purification apparatus 1 has a higher dissolved-oxygen concentration than usual. In addition, the reduction of bacteria and organic matter in the solution also leads to an increase in the dissolved oxygen. The use of the nutrient solution having highly concentrated dissolved oxygen can improve the growth rate of the plants 268.

Furthermore, the nutrient solution flown out from the purification apparatus 1 contains a part of the ozone dissolved in the first chamber 16. The action of the ozone itself also can improve the growth rate of the plants 268.

Thus, with the use of the purification apparatus 1, the growth rate of the plants 268 can be improved using the plurality of factors, thereby achieving efficient hydroponic culture.

While the second embodiment of the present disclosure has been described hereinabove, the present embodiment is not limited thereto.

For example, the present embodiment has described the example of the seedling raising apparatus 201 provided with the three-stage cultivation shelves 264. However, the purification apparatus 1 can purify the nutrient solution regardless of the arrangement and numbers of the cultivation shelves 264 provided in the seedling raising apparatus 201. In addition, when high purification ability is required due to circumstances such as a large-sized seedling raising apparatus 201, the purification apparatus 1 can be obviously provided in plural numbers or the ultraviolet unit 32 may be provided in plural numbers in a large-sized purification container 11.

The present embodiment has described the example of purifying the nutrient solution, as an application example of the purification apparatus 1. However, the target to be purified by the purification apparatus 1 is not limited to nutrient solution.

Examples of the target to be purified by the purification apparatus 1 include water for raising or breeding fish and shellfish and water for domestic use, such as water for drinking and water for bathing.

For example, when using the purification apparatus 1 to purify water for raising or breeding of fish and shellfish, there may be employed a structure without the purification container 11, namely, a structure including the ultraviolet unit 32, the introduction pipe 52, the pump 53, the exhaustion pipe 55, the air stone 56, and as needed, the photocatalyst member 33.

In this case, for example, the ultraviolet unit 32, the exhaustion pipe 55, the air stone 56, and as needed, the photocatalyst member 33 may be arranged in the water, and the pump 53 may be arranged so as to supply outside air through the introduction pipe 52.

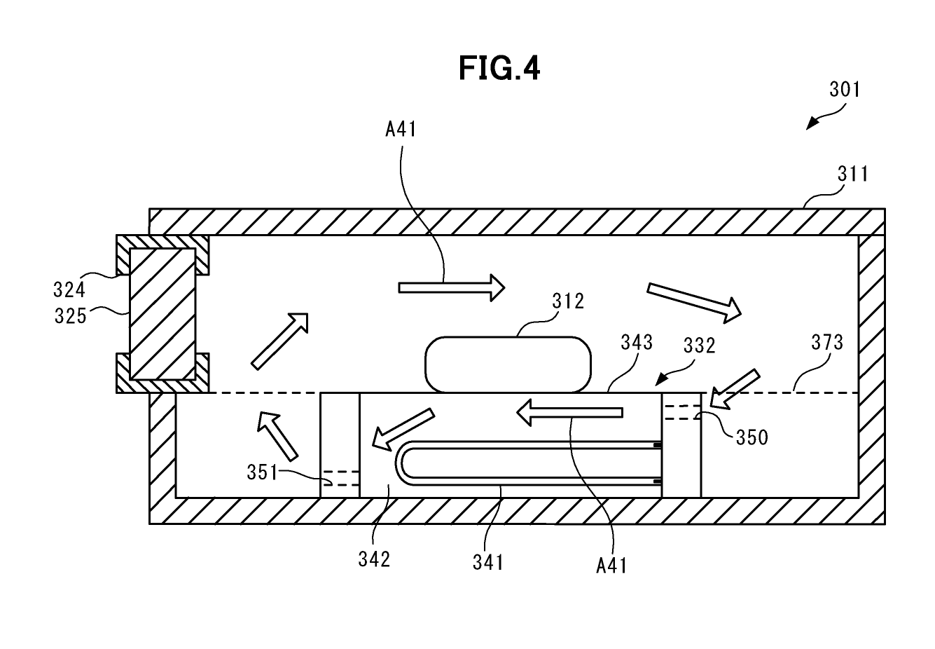

The target to be purified by the purification apparatus 1 may be, for example, a solid. FIG. 4 depicts an example of a purification apparatus 301 purifying a purification target 312 placed in a purification container 311 by exposure to ozone for a certain length of time. In this example, the purification container 311 is sealed, except for a ventilator 324 provided with a filter 325. An ultraviolet unit 332 disposed in the purification container 311 includes a cylindrical sealed accommodator 343 and an ultraviolet lamp 341 disposed therein.

The accommodator 343 is arranged horizontally (sideways) in a longitudinal direction thereof. On walls of the accommodator 343 opposing to each other in the longitudinal direction thereof are provided an oxygen introducing portion 350 and an ozone exhausting portion 351. The ozone exhausting portion 351 is located lower than the oxygen introducing portion 350. The purification target 312 is mounted on a placement base 373 having a plurality of holes for allowing ozone to pass through, which is provided in the purification container 311.

Although not depicted in the drawing, there may be disposed a fan or the like for forcing a gas to flow in order to cause ozone to flow in the purification space inside the purification container 311 and also in order to introduce oxygen or air into an accommodation space 342 inside the accommodator 343 to generate and exhaust ozone.

In this way, ozone is generated by the ultraviolet unit 332 and flows in a direction indicated by an arrow A41 in FIG. 4 to circulate in the purification space. Accordingly, the purification target 312 is exposed to the ozone by maintaining the purification container 311 accommodating the purification target 312 in a sealed state for a certain length of time. In addition, the purification target 312 is purified by irradiation with an ultraviolet ray having passed through a transmission portion of the ultraviolet unit 332. After the certain length of time, the ozone in the purification container 311 is exhausted outside by a not-shown fan or the like through the filter 324 and then a lid of the purification container 311 is opened, whereby the purification target 312 can be taken out safely without causing an operator to inhale the ozone.

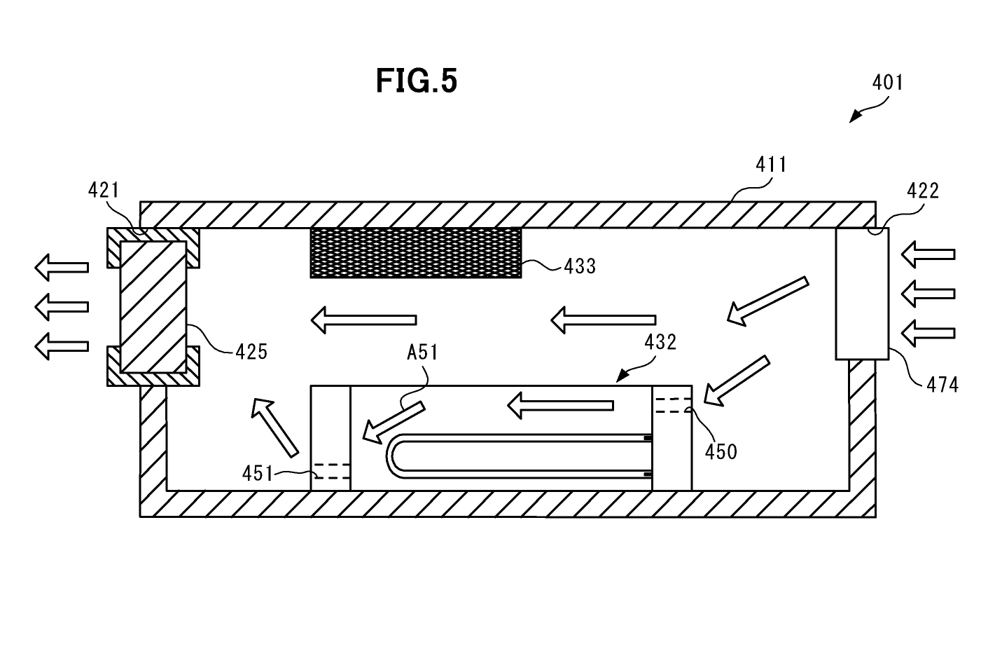

The target to be purified by the purification apparatus 1 may be, for example, a gas. FIG. 5 depicts an example of purifying air as a purification target by a purification apparatus 401. As depicted in the drawing, an ultraviolet unit 432 is arranged sideways in a purification container 411, like the ultraviolet unit 332 described with reference to FIG. 4. The purification container 411 is sealed such that a purification space inside the purification container 411, except for an inflow inlet 422 and an outflow outlet 421, does not communicate with an outside of the apparatus. The inflow inlet 422 is formed on a wall of the purification container 411 opposing to an oxygen introducing portion 450 of the ultraviolet unit 432 and has a fan 474. Outside air continuously flows into the purification space through the fan 474. The outflow outlet 421 is formed on a wall of the purification container 411 opposing to an ozone exhausting portion 451 of the ultraviolet unit 432 and has a filter 425. The purification container 411 further includes a photocatalyst member 433 on an inner wall thereof located above the ultraviolet unit 432.

With such a structure as described above, outside air flown into the purification space from the inflow inlet 422 is purified by the actions of ozone, at least one ultraviolet ray, the photocatalyst member 433, flows as indicted by an arrow A51 in FIG. 5, and then flows out from the outflow outlet 421. Since the outflow outlet 421 is provided with the filter 425, ozone is decomposed by the filter and flows out. Thus, air can be safely purified.

While some embodiments and modifications of the present disclosure have been described hereinabove, the disclosure is not limited thereto and includes, for example, modes made by combining each of the embodiments and each of the modifications as needed and technical ranges equivalent thereto.

A part or all of the above-described embodiments may be described as in the following supplementary notes but are not limited thereto.

(Supplementary Note 1)

A purification apparatus comprising:
an ultraviolet lamp emitting an ultraviolet ray with a wavelength having ozone generating effect and an ultraviolet ray with a wavelength having sterilizing effect;
an accommodator including a transmission portion that transmits the ultraviolet ray with a wavelength having sterilizing effect and accommodating the ultraviolet lamp in an accommodation space inside the accommodator;
a gas supplier in which a gas supply opening for supplying an oxygen-containing gas into the accommodation space is formed in the accommodator; and
an exhauster in which an exhaustion opening for exhausting ozone generated in the accommodation space due to the ozone generating effect of the ultraviolet ray emitted by the ultraviolet lamp is formed in the accommodator.

(Supplementary Note 2)

The purification apparatus according to the supplementary note 1, wherein the ultraviolet lamp is a discharge lamp in which metal vapor is sealed.

(Supplementary Note 3)

The purification apparatus according to the supplementary note 2, wherein the ultraviolet lamp is a cold cathode lamp.

(Supplementary Note 4)

The purification apparatus according to any one of the supplementary notes 1 to 3, wherein a material of a discharge tube included in the ultraviolet lamp or a material of the transmission portion is synthetic quartz, molten quartz, ozoneless quartz, or borosilicate glass containing at least one of Al, Na, K, Li, Ca, and Ba.

(Supplementary Note 5)

The purification apparatus according to any one of the supplementary notes 1 to 4, further comprising a purification container forming a purification space in which a purification target is arranged or flows, wherein
the transmission portion is arranged in the purification space.

(Supplementary Note 6)

The purification apparatus according to the supplementary note 5, comprising an ozone discharger that discharges ozone exhausted from the exhauster into the purification space, wherein
the purification container forms the purification space including a first chamber and a second chamber that are separated from each other and a flow channel communicating the first chamber with the second chamber;
the ozone discharger is provided in the first chamber; and
the ultraviolet lamp and the transmission portion of the accommodator are provided in the second chamber.

(Supplementary Note 7)

The purification apparatus according to the supplementary note 6, wherein the ozone discharger comprises an air stone or a micro-bubble generating device that forms the ozone emitted from the exhauster into air bubbles and discharges the ozone air bubbles into the purification space.

(Supplementary Note 8)

The purification apparatus according to the supplementary note 6 or 7, wherein the purification container comprises:
a communicator communicating the first chamber with an outside; and
a filter provided in the communicator to decompose ozone.

(Supplementary Note 9)

The purification apparatus according to any one of the supplementary notes 6 to 8, wherein the purification container comprises:
an inflow inlet allowing a fluid as the purification target to flow into the first chamber from the outside; and
an outflow outlet allowing the fluid to flow out from the second chamber to the outside.

(Supplementary Note 10)

The purification apparatus according to any one of the supplementary notes 1 to 9, further comprising a photocatalyst member provided around the accommodator and causing a photocatalytic reaction due to irradiation of an ultraviolet ray or visible light.

(Supplementary Note 11)

The purification apparatus according to the supplementary note 10, wherein the photocatalyst member includes a titanium oxide material on a surface thereof.

(Supplementary Note 12)

A purification method comprising:
a step of generating an ultraviolet ray with a wavelength having ozone generating effect and an ultraviolet ray with a wavelength having sterilizing effect;
a step of generating ozone by irradiating an oxygen-containing gas with the generated ultraviolet rays;
a step of supplying the generated ozone to a purification target; and
a step of irradiating the purification target with the generated ultraviolet rays.

The present disclosure is based on Japanese Patent Application No: 2011-249463, the entire specification, claims, and drawings of which are incorporated herein by reference.

### REFERENCE SIGNS LIST

1, 301, 401 Purification apparatus
11, 311, 411 Purification container
12 Purification tank
13 Lid
14 Partition plate
15 Flow channel
16 First chamber
17 Second chamber
21, 421 Inflow inlet
22, 422 Outflow outlet
23 Conduit portion
24, 324 Ventilator
25, 325, 425 Filter
26 Filter attachment unit
31 Ultraviolet unit installation portion
32, 332, 432 Ultraviolet unit
33, 433 Photocatalyst member
36 Engagement portion
41, 341 Ultraviolet lamp
42, 342 Accommodation space
43, 343 Accommodator
45 U-shaped tube
46 Electrode
50, 350, 450 Oxygen introducing portion
51, 351, 451 Ozone exhausting portion
52 Introduction pipe
53 Pump
55 Exhaustion pipe
56 Air stone
201 Seedling raising apparatus
261 Support member
262 Nutrient solution tank
263 Nutrient solution pump
264 Cultivation shelf
265 Lighting fixture
266 Purified-nutrient solution pipe
267 Plant placement plate
268 Plant
269 Nutrient solution conduit
270 Contaminated-solution pipe
271 Pressurized-solution ejecting portion
272 Injection portion
373 Placement base
474 Fan

1. A purification apparatus, comprising:
an ultraviolet lamp emitting an ultraviolet ray with a wavelength having ozone generating effect and an ultraviolet ray with a wavelength having sterilizing effect;
an accommodator including a transmission portion that transmits the ultraviolet ray with a wavelength having sterilizing effect and accommodating the ultraviolet lamp in an accommodation space inside the accommodator;
a gas supplier supplying an oxygen-containing gas into the accommodation space; and
an exhauster exhausting ozone generated in the accommodation space due to the ozone generating effect of the ultraviolet ray emitted by the ultraviolet lamp.

2. The purification apparatus according to claim 1, wherein
the ultraviolet lamp is a discharge lamp in which metal vapor is sealed.

3. The purification apparatus according to claim 2, wherein
the ultraviolet lamp is a cold cathode lamp.

4. The purification apparatus according to claim 1, wherein
a material of a discharge tube included in the ultraviolet lamp or a material of the transmission portion is synthetic quartz, molten quartz, ozoneless quartz, or borosilicate glass containing at least one of Al, Na, K, Li, Ca, and Ba.

5. The purification apparatus according to claim 1, further comprising a purification container forming a purification space in which a purification target is arranged or flows, wherein
the transmission portion is arranged in the purification space.

6. The purification apparatus according to claim 5, comprising an ozone discharger that discharges ozone exhausted from the exhauster into the purification space, wherein
the purification container forms a purification space including a first chamber and a second chamber that are separated and a flow channel communicating the first chamber with the second chamber;
the ozone discharger is provided in the first chamber; and
the ultraviolet lamp and the transmission portion of the accommodator are provided in the second chamber.

7. The purification apparatus according to claim 6, wherein
the ozone discharger comprises an air stone or a micro-bubble generating device that
forms the ozone exhausted from the exhauster into air bubbles and discharges the ozone air bubbles into the purification space.

8. The purification apparatus according to claim 6, wherein
the purification container comprises:
a communicator communicating the first chamber with an outside; and
a filter provided in the communicator to decompose ozone.

9. The purification apparatus according to claim 1, wherein
the purification container comprises:
an inflow inlet allowing a fluid as the purification target to flow into the first chamber from the outside; and
an outflow outlet allowing the fluid to flow out from the second chamber to the outside.

10. The purification apparatus according to claim 1, further comprising a photocatalyst member provided around the accommodator and causing a photocatalytic reaction due to irradiation of an ultraviolet ray or visible light.

11. A purification method comprising:
a step of generating an ultraviolet ray with a wavelength having ozone generating effect and an ultraviolet ray with a wavelength having sterilizing effect;
a step of generating ozone by irradiating an oxygen-containing gas with the generated ultraviolet rays;
a step of supplying the generated ozone to a purification target; and
a step of irradiating the purification target with the generated ultraviolet rays.

12. The purification apparatus according to claim 2, wherein
a material of a discharge tube included in the ultraviolet lamp or a material of the transmission portion is synthetic quartz, molten quartz, ozoneless quartz, or borosilicate glass containing at least one of Al, Na, K, Li, Ca, and Ba.

13. The purification apparatus according to claim 3, wherein
a material of a discharge tube included in the ultraviolet lamp or a material of the transmission portion is synthetic quartz, molten quartz, ozoneless quartz, or borosilicate glass containing at least one of Al, Na, K, Li, Ca, and Ba.

14. The purification apparatus according to claim 2, further comprising a purification container forming a purification space in which a purification target is arranged or flows, wherein
the transmission portion is arranged in the purification space.

15. The purification apparatus according to claim 3, further comprising a purification container forming a purification space in which a purification target is arranged or flows, wherein
the transmission portion is arranged in the purification space.

16. The purification apparatus according to claim 4, further comprising a purification container forming a purification space in which a purification target is arranged or flows, wherein
the transmission portion is arranged in the purification space.

17. The purification apparatus according to claim 7, wherein
the purification container comprises:
a communicator communicating the first chamber with an outside; and
a filter provided in the communicator to decompose ozone.

18. The purification apparatus according to claim 2, further comprising a photocatalyst member provided around the accommodator and causing a photocatalytic reaction due to irradiation of an ultraviolet ray or visible light.

19. The purification apparatus according to claim 3, further comprising a photocatalyst member provided around the accommodator and causing a photocatalytic reaction due to irradiation of an ultraviolet ray or visible light.

20. The purification apparatus according to claim 4, further comprising a photocatalyst member provided around the accommodator and causing a photocatalytic reaction due to irradiation of an ultraviolet ray or visible light.
